(19) United States
(12) Patent Application Publication
(10) Pub. No.: US 2024/0407880 A1
(43) Pub. Date: Dec. 12, 2024

(54) OPTICAL ELEMENT FOR SURGICAL MICROSCOPES AND METHOD FOR PRODUCING SAME, AND MEDICAL OPTICAL DEVICE

(71) Applicant: TOPCON CORPORATION, Tokyo (JP)

(72) Inventors: Takuro OKUBO, Itabashi-ku, Tokyo (JP); Takashi TAKAHASHI, Itabashi-ku, Tokyo (JP)

(73) Assignee: TOPCON CORPORATION, Tokyo (JP)

(21) Appl. No.: 18/699,892

(22) PCT Filed: Aug. 12, 2022
(86) PCT No.: PCT/JP2022/030836

§ 371 Date: Apr. 10, 2024

(30) Foreign Application Priority Data
Oct. 11, 2021 (JP) 2021-166808

(51) Int. Cl. A61B 90/20; G02B 1/115; G02B 1/18; G02B 21/00

(52) U.S. Cl. CPC A61B 90/20; G02B 1/115; G02B 1/18; G02B 21/0012

(57) ABSTRACT

The optical element for surgical microscopes having antifogging properties, antibacterial properties, and durability against autoclave treatment is provided, the optical element including a substrate, an antireflection coating arranged on the substrate, and an antifouling coating layer containing acrylic polymer arranged on the antireflection coating, in which C—H bonding of carbon of the end of the surface of the antifouling coating layer is substituted by C—OSi(OH)₃ by a combustion chemical vapor deposition method. A method for production thereof includes steps of coating a solution containing alkoxysilane having an acrylic group on the antireflection coating so as to form the antifouling coating layer, and substituting C—H bonding of carbon of the end of the surface of the antifouling coating layer by C—OSi(OH)₃ by a combustion chemical vapor deposition method using silane based gas.

### TECHNICAL FIELD

The present invention relates to an optical element for surgical microscopes, to a method for producing the same, and to a medical optical device, and in particular, relates to an optical element for surgical microscopes and a medical optical device in which good antifogging properties, antibacterial properties, and durability against autoclaving treatment that can be maintained for a long time.

### BACKGROUND ART

In medical optical devices, particularly in an operating microscope system (OMS), since an eye lens used in an operation on an eye will be close to a patient, there are situations in which the lens may be fogged by exhaled breath, and the operation should be stopped, and a situations in which bacteria may adhere to the lens in a case in which a hydrophilic coating is arranged on the surface to prevent fogging of the lens.

To solve such problems, an optical element has been proposed in which an antireflection coating is arranged on glass substrate and antifouling coating layer comprising acrylic polymer containing at least one selected from alcohols and esters is layered on the antireflection coating (for example, see Patent Document 1). According to this technique, superior antifogging properties can be imparted by making the antifouling coating layer hydrophilic, and in addition, antibacterial properties can be improved without deteriorating antifogging properties by containing at least one selected from alcohols and esters.

However, since it is to be used for medical devices, sterilizing treatment by autoclaving at not less than 130° C., at not less than 0.32 atm, and for not less than 10 minutes is required. Therefore, in the optical element disclosed in Patent Document 1, an antifouling coating layer consisting of organic material may be decomposed, and thus, it has a problem of durability.

Furthermore, with respect to an optical element including a hydrophilic antifouling coating layer, a method for maintaining antifogging properties on the surface of the optical element is disclosed, in which wet wiping on the surface of the optical element is performed after high temperature and high pressure treatment (for example, see Patent Document 2).

However, in this method, there is a problem in which the wet wiping process and recoating process of an antifouling coating layer and the like are required after each autoclaving treatment at high temperature and high pressure, and thus, processes are complicated.

Furthermore, a combustion chemical vapor deposition (CCVD) technique is known in which silane-based gas is combusted by a flame burner so as to generate nanoscale silicon oxide fine particles, it is adhered on the surface of a substrate so as to generate Si—OH bonding on the surface thereof, so that antifogging properties are imparted.

However, there is a problem in that Si—OH does not strongly adhere to the surface of the substrate since it is an inorganic material, Si—OH detaches easily, and antifogging properties are lost.

The Patent Documents are as follows.
Patent Document 1: Japanese Patent Publication No. 6799932
Patent Document 2: Japanese Unexamined Patent Application, Publication No. 2016-64260

### SUMMARY OF INVENTION

The present invention was completed in view of the above circumstances, and an object of the present invention is to provide an optical element for surgical microscopes and a medical optical device which withstand autoclaving treatment and enable maintaining high antifogging properties and antibacterial properties over a long term.

The present invention is to accomplish the above object, and the first aspect according to the invention is an optical element for surgical microscopes, comprising: a substrate; an antireflection coating arranged on the substrate; and an antifouling coating layer containing acrylic polymer arranged on the antireflection coating, in which bonding to carbon at the end of the surface of the antifouling coating layer is C—OSi(OH)₃.

According to the optical element for surgical microscopes of the present invention, since acrylic polymer is used in the antifouling coating layer layered on the antireflection coating, the antifouling coating layer can be made hydrophilic, and superior antifogging properties can be imparted. Furthermore, since bonding to carbon of the end derived from the acrylic polymer at the most surface side of the antifouling coating layer is C—OSi(OH)₃, it may not be decomposed even by autoclaving treatment at high temperature and high pressure, and durability is improved.

The second aspect according to the invention is the optical element for surgical microscopes according to the first aspect, wherein the antifouling coating layer contains at least one selected from alcohols and esters.

According to the optical element for surgical microscopes of the present invention, by containing at least one selected from alcohols and esters, antibacterial properties can be maintained without deteriorating antifogging properties.

The third aspect according to the invention is the optical element for surgical microscopes according to the second aspect, wherein the alcohols is phenoxyethanol.

The fourth aspect according to the invention is the optical element for surgical microscopes according to the third aspect, wherein content of the phenoxyethanol is in a range of 1 wt % to 5 wt %.

According to the aspect, by setting the content of phenoxyethanol in a range of 1 wt % to 5 wt %, superior antibacterial properties can be imparted without adversely affecting optical properties of the optical element.

The fifth aspect according to the invention is the optical element for surgical microscopes according to any one of the first to forth aspects, wherein the antireflection coating is formed by layering, from the substrate side, a first high refraction index layer comprising ditantalum pentoxide or hafnium dioxide, a first low refraction index layer comprising silicon oxide, and a second high refraction index layer comprising ditantalum pentoxide or hafnium dioxide.

The sixth aspect according to the invention is the optical element for surgical microscopes according to the fifth aspect, wherein the antireflection coating is formed by layering, on the second high refraction index layer, a second low refraction index layer comprising silicon oxide and a third high refraction index layer comprising ditantalum pentoxide or hafnium dioxide.

The aspect defines the antireflection coating, and by forming the antireflection coating of the above structure, low reflective index can be achieved against light having wavelengths in a range of 420 nm to 750 nm.

The seventh aspect according to the invention is the optical element for surgical microscopes according to any one of the first to sixth aspects, wherein the substrate is lanthanum based glass or synthetic quartz glass.

The eighth aspect according to the invention is the optical element for surgical microscopes according to any one of the first to seventh aspects, wherein coating thickness of the antifouling coating layer is in a range of 1 nm to 10 nm.

According to this aspect, by setting the coating thickness of the antifouling coating layer in the above range, the antifouling coating layer can be layered without adversely affecting optical properties of the optical element.

The ninth aspect according to the invention is a method for producing the optical element for surgical microscopes according to the first aspect, comprising steps of: forming the antireflection coating on the substrate; coating solution containing alkoxysilane having acrylic group on the antireflection coating so as to form the antifouling coating layer; and substituting C—H bonding of carbon of the end of the surface of the antifouling coating layer by C—OSi(OH)₃ by a combustion chemical vapor deposition method using silane based gas.

The tenth aspect according to the invention is a medical optical device comprising the optical element for surgical microscopes according to any one of the first to eighth aspects.

Since the above mentioned optical element for surgical microscopes has superior antifogging properties, antibacterial properties and durability against autoclaving treatment, it can be appropriately employed in a medical optical device.

According to the optical element of the present invention, by layering an antifouling coating layer comprising acrylic polymer on the surface and by substituting C—H bonding of carbon of an end of the surface side by C—OSi(OH)₃, antifogging properties, antibacterial properties, and durability against autoclave treatment can be realized. Furthermore, since this optical element can be prevented from being fogged during a surgical operation and has antibacterial effect, it can be appropriately employed in a medical surgical device.

### BRIEF DESCRIPTION OF DRAWINGS

FIG. 1 is a side cross sectional view showing a layered condition of the optical element for surgical microscopes of the first Embodiment.

FIG. 2 is a side cross sectional view showing a layered condition of the optical element for surgical microscopes of the second Embodiment.

FIG. 3 is a side cross sectional view schematically showing the antifouling coating layer before performing surface treatment by combustion chemical vapor deposition method.

FIG. 4 is a side cross sectional view schematically showing the antifouling coating layer after performing surface treatment by combustion chemical vapor deposition method.

FIG. 5 is a schematic view showing one example of an outer appearance structure of a medical optical device.

### EMBODIMENTS OF INVENTION

Hereinafter the optical element for surgical microscopes and the medical optical device according to the present invention are explained with reference to the drawings.

### First Embodiment

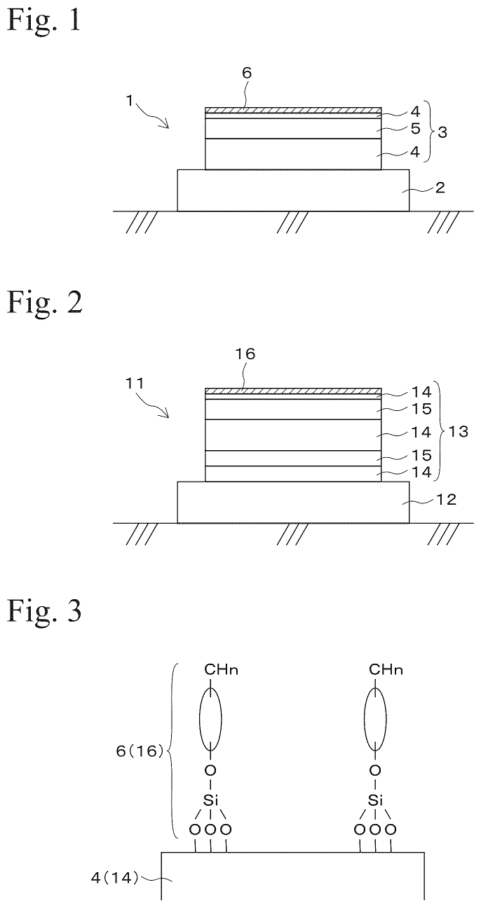

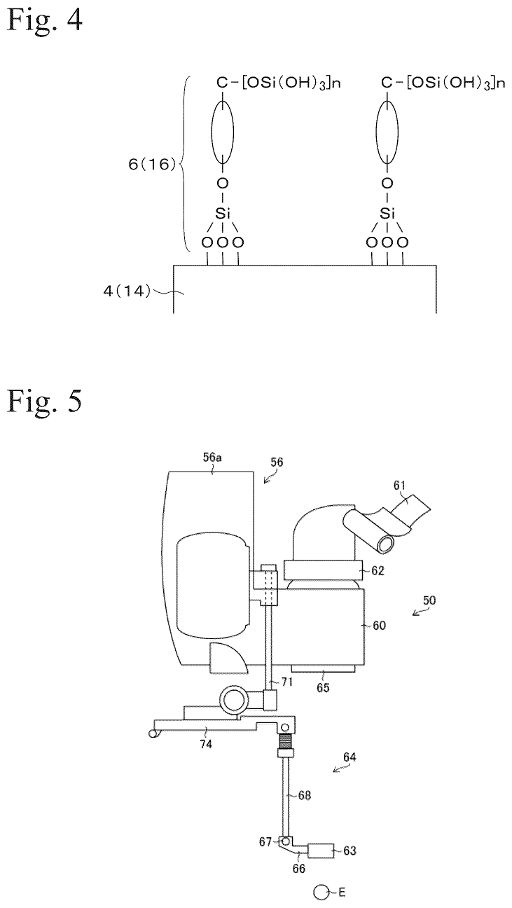

FIG. 1 is an explanatory view showing a layered condition of the optical element for surgical microscopes of the first Embodiment. In the optical element 1 for surgical microscopes of the first Embodiment, a first high refractive index layer 4 comprising ditantalum pentoxide, a first low refractive index layer 5 comprising silicon oxide (SiO₂), and a second high refractive index layer 4 comprising ditantalum pentoxide, as an antireflection coating 3, are layered on a substrate 2, and an antifouling coating layer 6 having thickness of an extent not adversely affecting optical properties is layered thereon.

As the substrate 2, a material typically used as an optical element for surgical microscopes can be selected, and lanthanum based glass LAH53, synthetic quartz glass or the like is used as the material.

The high refractive index layer 4 and the low refractive index layer 5 are not limited to these, and a combination of the layers can be selected freely as long as they have different refractive index each other. For example, as another selectable material, hafnium dioxide (HfO₂) can be layered instead of ditantalum pentoxide as the high refractive index layer 4.

A film formation of the antireflection coating 3 on the substrate can be performed by selecting any film forming method of an ion assist method, vacuum vapor deposition method, and spattering method to form each layer of the first high refractive index layer 4, the first low refractive index layer 5, and the second high refractive index layer 4.

Formation of the antifouling coating layer 6 on the antireflection coating 3 is performed by coating a solution of alkoxysilane including an acrylic group on the surface of the antireflection coating 3 (one solution coating), and then promoting a hydrolyzation reaction. Here, the “alkoxysilane including acrylic group” in the present invention means a compound in which one of the H of silane (SiH₄) is substituted by a “Si—O— acrylic group” or a “Si—O-(carbon chain including acrylic group)”. Then, 1 to 3H of the remainder in silane is or are substituted by an alkoxylic group (RO—).

Monoalkoxysilane, dialkoxysilane, and trialkoxysilane in which acrylic group or carbon chain including acrylic group is bonded to Si via O and one to three bonds of the remainder of Si is or are substituted by an alkoxyl group can be mentioned, and among these, “one acrylic group substituted+trialkoxysilane” in which in contrast three bonds to an acrylic group in Si are substituted by an alkoxylic group is desirable since networks of Si—O are formed plentifully with respect to the antireflection coating, and bonds may be strong. Alternatively, a mixture thereof can be used.

An R-group of an alkoxyl group, methyl, ethyl, propyl, or the like, which is conventionally known can be used. It is desirable that concentration of alkoxysilane including an acrylic group in solution be 0.1 to 5.0%. Methanol, ethanol or the like can be used as a solvent.

By coating treatment of the antifouling coating layer, a hydroxyl group may be generated by desorption of alkoxyl group, the hydroxyl group may be bonded to the surface of the antireflection coating, and siloxane bonding between the antireflection coating and the antifouling coating layer may be generated as “(antireflection coating-O)₃—Si—O-acrylic group (or carbon chain including acrylic group)” as shown in FIG. 3, so that a structure in which Si is bonded to the surface of the high refractive index layer 4 via O may be generated.

Preparation of alkoxysilane including acrylic group is omitted since it is a conventional technique using a silicon compound and acrylic polymer. After coating alkoxysilane including an acrylic group, as shown in FIG. 3, a structure is completed in which an organic group (carbon chain) derived from an acrylic polymer is bonded to the Si—O—. It should be noted that a part shown by an ellipse shape in FIG. 3 means a structure in which a freely selected carbon chain derived from an acrylic polymer is omitted. With respect to the antireflection coating, for example, the structure is like “—Si—O—CH₂— . . . —CH₃”. It is desirable that this carbon chain has a carbon number of several hundreds to several thousands. Instead of C—C single bonding in this carbon chain, double bonding and triple bonding can be contained at an arbitrary location. Furthermore, an OH group can be bonded instead of H at an arbitrary location in this carbon chain. The carbon chain can be a straight chain or a branched chain. In —CHₙ at the end of most surface side, n is a natural number 1 to 3, and they indicate —CH₃, ═CH₂ and ═CH in a case in which the end is a single bond, double bond, or triple bond, respectively. In addition, —CHₐ(OH)_{b} (here, a+b=3), ═CH_{c}(OH)_{d} (here, c+d=2) and —COH in which one to all three H are substituted by OH can be selected.

In the present invention, instead of alkoxysilane having a monosubstituted acrylic group, an alkoxysilane having a monosubstituted betaine structure, a multivalent alcohol structure, an amide structure, and a nonionic structure can be used. In a case in which alkoxysilane having these structures at one bonding of Si and having an alkoxyl group at the remaining one to three bonds is used, similar to the case of an acrylic group explained so far, the end of the Si side forms a siloxane bond at the antireflection coating, and then, with respect to C—H and C—OH of the end of the surfacemost side of the antifouling coating layer, a combustion chemical vapor deposition method mentioned below is performed.

As a raw material acrylic polymer used while preparing alkoxysilane including an acrylic group, any water soluble acrylic polymer and water insoluble acrylic polymer can be used, and a polymer or the like can be mentioned in which an acrylic ester or methacrylic ester including a straight or branched alkyl group with a carbon number not greater than 10 is contained as a component. Practically, an acrylic polymer of acrylic esters such as methyl acrylate, ethyl acrylate, or butyl acrylate can be used. As the acrylic polymer, one kind can be used alone, or multiple kinds can be used in a mixture.

By forming the antifouling coating layer 6 using alkoxysilane including an acrylic group, hydrophilic properties can be imparted to the antifouling coating layer 6. If the antifouling coating layer 6 is hydrophilic, water drops adhering on the antifouling layer 6 may be wet and spread on the surface, the optical element 1 for surgical microscopes can be prevented from being fogged. As an indication showing hydrophilic properties, a contact angle can be used, and it is desirable that the contact angle be not greater than 10°. It is desirable that the thickness of the physical coating of the antifouling coating layer 6 be 1 nm to 10 nm.

When forming the antifouling coating layer 6, it is desirable that alkoxysilane including an acrylic group including at least one selected from alcohols and esters be used for formation, because antibacterial properties may be improved.

As an alcohol, phenoxyethanol can be used. As an ester, paraoxybenzoic ester can be used, and practically, methylparaben, ethylparaben, propylparaben, isopropylparaben, butylparaben, isobutyl paraben, benzylparaben or the like can be mentioned. In particular, it is desirable to use phenoxyethanol. In addition, it is desirable that the content of alcohols and esters be 1 wt % to 5 wt % with respect to weight of alkoxysilane including an acrylic group.

As a method to coat the solution, a spin coating method may be mentioned. For example, coating can be completed by twice performing coating at 1500 rpm and 20 seconds. In addition, drying can be performed at a normal temperature, and alternatively, at 85° C. heating for 1 hour. In FIG. 3, the reference numeral 6 corresponds to the antifouling coating layer.

Next, the combustion chemical vapor deposition (hereinafter referred to as CCVD) treatment is performed for C—H bonding (or C—OH bonding) of the end of the surfacemost side of the antifouling layer 6. A silane based gas such as monosilane or disilane, hydrocarbon gas and oxygen are supplied, nanoscale silicon oxide particles may be generated by an oxidizing flame by a flame burner, and the nanoscale silicon oxide particles adhere on the surface to be coated at a gathered density close to a membrane, so as to improve the surface. According to this CCVD treatment, the —C—H (or —C—OH) bonding at the end becomes —C—[OSi(OH)₃]ₙ (n=1 to 3).

The conditions of the CCVD treatment are such that the above gas mixture is kept at not less than 1000° C. for about several seconds.

If an end of an organic group is C—Hₙ, the carbon chain may be decomposed by autoclaving treatment at 132° C. and 0.32 MPa and for 10 minutes for example; however, it is clear that if the bonding is C—[OSi(OH)₃]ₙ, it may not be decomposed, and durability may be improved.

As a practical example of a layer structure of the optical element 1 for surgical microscopes, for example, the following compositions and physical coating thickness shown in Table 1 can be employed.

TABLE 1

| | | Physical coating |
|---|---|---|
| Layer | Material | thickness (nm) |
| Substrate | Lanthanum based glass | |
| First layer (First high | Ta₂O₅ | 110.57 to 111.57 |
| refractive index layer) | | |
| Second layer (First low | SiO₂ | 66.61 to 67.61 |
| refractive index layer) | | |
| Third layer (Second high | Ta₂O₅ | 4.5 to 5.5 |
| refractive index layer) | | |
| Antifouling layer | Acrylic polymer + | 1 to 10 |
| | phenoxyethanol | |

### Second Embodiment

FIG. 2 is a schematic diagram showing a condition of layering of an optical element for surgical microscopes of the second embodiment. An optical element 11 for surgical microscopes is formed by a substrate 12; a first high refractive index layer 14 of ditantalum pentoxide (Ta₂O₅), a first low refractive index layer 15 of silicon oxide (SiO₂), a second high refractive index layer 14 of ditantalum pentoxide (Ta₂O₅), a second low refractive index layer 15 of silicon oxide (SiO₂), and a third high refractive index layer 14 of ditantalum pentoxide (Ta₂O₅) layered on the substrate 12 as an antireflection coating 13; and an antifouling coating layer 16 having thickness of degree not affecting optical properties on the antireflection coating 13. That is, the optical element 11 for surgical microscopes of the second embodiment differs from the optical element 1 for surgical microscopes of the first embodiment because the antireflection coating 13 includes five layers. It should be noted that, similar to in the first embodiment, hafnium dioxide (HfO₂) can be layered instead of ditantalum pentoxide.

As the substrate 12, similar to in the first embodiment, the lanthanum based glass LAH 53 or synthetic quartz glass can be used as a material.

A method for forming a coating of the antireflection coating 13 and a material and method for forming the antifouling coating layer 16 can be the same as those in the optical element for surgical microscopes of the first embodiment, and explanation thereof is omitted.

As a practical example of a layering structure of the optical element 11 for surgical microscopes, for example, the compositions and physical coating thicknesses shown in the following Table 2 may be mentioned.

TABLE 2

| | | Physical coating |
|---|---|---|
| Layer | Material | thickness (nm) |
| Substrate | Lanthanum based glass | |
| First layer (First high | Ta₂O₅ | 11.70 to 13.90 |
| refractive index layer) | | |
| Second layer (First low | SiO₂ | 36.05 to 37.25 |
| refractive index layer) | | |
| Third layer (Second high | Ta₂O₅ | 116.11 to 117.11 |
| refractive index layer) | | |
| Forth layer (Second low | SiO₂ | 74.15 to 75.15 |
| refractive index layer) | | |
| Fifth layer (Third high | Ta₂O₅ | 2.66 to 3.66 |
| refractive index layer) | | |
| Antifouling layer | Acrylic polymer + | 1 to 10 |
| | phenoxyethanol | |

It should be noted that the embodiment in which the high refractive index layer 4 and the low refractive index layer 5 are alternately layered to form three layers is shown in the first embodiment and the embodiment in which the high refractive index layer 14 and the low refractive index layer 15 are alternately layered to form five layers is shown in the second embodiment as an antireflection layer; however, the present invention is not limited to these embodiments, and the number of layers of the high refractive index layer and the low refractive index layer is not limited in particular, as long as the lowest layer and the highest layer in the antireflection layer are high refractive index layers.

### Medical Optical Device

As one example of the medical optical device including the above optical element, a microscope for an operator used in a surgical microscope is explained. FIG. 5 is a schematic drawing showing one example of an outer appearance of the microscope for an operator. The above mentioned optical element can be desirably used as a forward-mounted lens.

The surgical microscope 50 includes a support pillar (which is not shown) and the microscope 56 for operator which is hung on an arm. An operator operates a foot switch (which is not shown) by foot so that the microscope for operator 56 is moved three-dimensionally.

In a lens tube unit 60 of the microscope 56 for operator, kinds of optical systems and driving systems are contained. On the upper part of the lens tube unit 60, an inverter unit 62 is arranged. The inverter unit 62 is an optical unit in which an inverted image is converted into an right-side up image. An eye contact unit 61 is arranged at the upper part of the inverter unit 62. An eye lens is arranged at the eye contact unit 61 and an objective lens 65 is arranged at the lower end of the lens tube unit 60.

An upper end part of a holding arm 64 is connected to the microscope 56 for operator. The forward-mounted lens 63 is held at an lower end part of the holding arm 64. The forward-mounted lens 63 focuses illumination light so as to illuminate the inside of an eye E to be operated on. As the forward-mounted lens 63, multiple lenses having mutually different refractive powers (for example, 40D, 80D, 120D or the like) are prepared, and these are alternatively attached to the holding arm 64.

The upper end part of the holding arm 64 is contacted pivotably along an up and down direction. According to this, the forward-mounted lens 63 is made so as to enable being inserted and detached at a location between the eye E to be operated on and the objective lens 65. A location in which the forward lens is inserted (operation location) is a location on the optical axis of the objective lens 65, and that a location between a forward focus position of the objective lens 65 and the eye E to be operated on. FIG. 5 shows conditions in which the forward-mounted lens 63 is arranged at the operation location between the eye E to be operated on and the objective lens 65.

The forward-mounted lens 63 is held by a holding plate 66 which is formed surrounding therearound, is connected to an arm unit 68 via a pivot 67, and is made rotatable around the pivot 67 as a center.

Furthermore, the microscope 56 for operator includes an elevating arm 71, and main body unit 56a of the microscope 56 for operator and a containing unit 74 which contains the holding arm 64 and the forward-mounted lens 63 are contained and are connected to the elevating arm 71. By moving the elevating arm 71 along an up and down direction, the forward-mounted lens 63 is also integrally moved. The containing unit 74 is formed so as to be attachable and detachable with respect to the elevating arm 71. This is because the forward-mounted lens 63 and the holding arm 64 are detached from the microscope 56 for operator when they are sterilized.

The forward-mounted lens 63 acts focusing illuminating light which comes from the objective lens 65 and introducing it into the inside of an eye. It is used when the forward-mounted lens 63 is contacted to the eye E to be operated on and the inside of the eye E to be operated on (retina, vitreum, or the like) is observed.

The forward mounted lens 63 is arranged at a location several millimeters from the eye E to be operated on, and then, the operation is performed. Therefore, there is a possibility that exhaled breath from a person who is operated will fog the forward mounted lens 63 and the operation will be stopped; however, by using the optical element of the present invention as the forward mounted lens, the forward mounted lens can be prevented from being fogged. Furthermore, it has superior antibacterial properties and durability in which the antifouling coating layer is not decomposed by a repeatedly performed autoclave treatment at high temperature and high pressure after the operation, it can be appropriately used as a medical optical device for a long term.

### EXAMPLES

Hereinafter the present invention is explained in detail with reference to Examples. However, the present invention is not limited to the following Examples, and the present invention is supported based on the entire disclosure of the description.

### Example 1, Comparative Example 1

As an optical element, the optical element 1 for surgical microscopes (including three layers of antireflection coatings) of the first embodiment shown in FIG. 1 was used. As an antifouling coating layer 6, trimethoxysilane having an acrylic group in which among all of bonds to Si, one bond was substituted was wet-coated on the antireflection layer 3. After coating, rinsing was performed using pure water so as to make a single molecular layer and to remove excess molecules. This was dried and washed using water to prepare Comparative Example 1. Furthermore, with respect to one which was prepared similarly as in Comparative Example 1, CCVD treatment was performed by silane based gas to treat C—H bonding of the end so as to prepare Example 1.

### Example 2, Comparative Example 2

Optical elements of Example 2 and Comparative Example 2 were prepared similarly to Example 1 and Comparative Example 1, respectively, except that trimethoxysilane having a betaine structure was used, in which among all of bonds to Si, one bond was substituted.

### Example 3 and Comparative Example 3

Optical elements of Example 3 and Comparative Example 3 were prepared similarly to Example 1 and Comparative Example 1, respectively, except that trimethoxysilane having a multivalent alcohol structure was used, in which among all of bonds to Si, one bond was substituted.

### Example 4 and Comparative Example 4

Optical elements of Example 4 and Comparative Example 4 were prepared similarly to Example 1 and Comparative Example 1, respectively, except that trimethoxysilane having an amide structure was used, in which among all of bonds to Si, one bond was substituted.

### Example 5 and Comparative Example 5

Optical elements of Example 5 and Comparative Example 5 were prepared similarly to Example 1 and Comparative Example 1, respectively, except that trimethoxysilane having a nonionic structure was used, in which among all of bonds to Si, one bond was substituted.

### Evaluation Method

Antifogging properties were confirmed at multiple steps before and after autoclaving treatment of the optical elements for surgical microscopes of the Examples and Comparative Examples, so as to evaluate durability against autoclaving treatment. Antifogging properties were performed as follows: exhaled breath test was performed toward the surface of the optical element under conditions of room temperature 25±2° C. and humidity 40±10 RH %, and fogging was confirmed by visual inspection. A sample in which fogging disappeared immediately and antifogging properties were superior was evaluated as A, a sample in which antifogging properties were uneven was evaluated as B, and a sample in which fogging did not disappear over the entire surface of the element was evaluated as C. The autoclaving treatment was performed at 132° C. and 0.32 MPa and for 10 minutes each time.

Table 3 shows evaluation for Comparative Examples. In Table 3, “CL (1), former” means that the test was performed before rinsing using pure water to make the coating single molecule, and “CL (1), latter” means that the test was performed after rinsing. “CL (2), former” means that the test was performed before wiping once by hand with a wipe immersed in pure water, and “CL (2), latter” means that the test was performed after wiping by hand.

TABLE 3

| | Before autoclave treatment | | After autoclave treatment | |
|---|---|---|---|---|
| | (once) | | (once) | |
| | CL (1), | CL (1), | CL (2), | CL (2), |
| | former | latter | former | latter |
| Comparative | A | A | B-C | B-C |
| Example 1 | | | | |
| Comparative | A | A | C | C |
| Example 2 | | | | |
| Comparative | A | A | C | C |
| Example 3 | | | | |
| Comparative | B | A | C | C |
| Example 4 | | | | |
| Comparative | A | A | C | C |
| Example 5 | | | | |

As shown in Table 3, in Comparative Example 4, although antifogging properties were inferior before rinsing using pure water to make a single-molecule coating, antifogging properties were improved after rinsing, and antifogging properties were A in all of Comparative Examples 1 to 5. However, after an autoclave treatment, antifogging properties were deteriorated, and antifogging properties were not recovered in spite of it being wiped by hand with a wipe. Subsequently, Table 4 shows evaluation regarding Examples.

TABLE 4

| | Before autoclave treatments | | After autoclave treatments | |
|---|---|---|---|---|
| | (10 times) | | (10 times) | |
| | CL (1), | CL (1), | CL (2), | CL (2), |
| | former | latter | former | latter |
| Example 1 | A | A | A | A |
| Example 2 | A | A | A | A |
| Example 3 | A | A | A | A |
| Example 4 | B | A | A | A |
| Example 5 | A | A | A | A |

As shown in Table 4, in Examples 1 to 5 of the present invention, antifogging properties were maintained after ten autoclaving treatments, and furthermore, it became clear that antifogging properties were maintained after wiping by hand with a wipe.

As explained so far, according to the optical element of the Examples, the optical element can have durability against autoclaving treatment and maintain antifogging properties. In addition, by adding antibacterial components in the antifouling coating layer, antibacterial properties can also be exhibited.

### EXPLANATION OF REFERENCE NUMERALS

1, 11: Optical element, 2, 12: substrate, 3, 13: antireflection coating, 4, 14: high refractive index layer, 5, 15: low refractive index layer, 6, 16: antifouling coating layer, 50: surgical microscope, 56: microscope for operator, 56a: main body unit, 60: lens tube unit, 61: eye lens unit, 62: inverter unit, 63: forward mounted lens, 64: holding arm, 65: objective lens, 66: holding plate, 67: pivot, 68: arm unit, 71: elevating arm, and 74: containing unit

1. An optical element, for surgical microscopes, comprising:
a substrate,
an antireflection coating arranged on the substrate, and
an antifouling coating layer containing acrylic polymer arranged on the antireflection coating,
wherein bonding to carbon of the end of the surface of the antifouling coating layer is C—OSi(OH)₃.

2. The optical element for surgical microscopes, according to claim 1, wherein the antifouling coating layer contains at least one selected from alcohols and esters.

3. The optical element for surgical microscopes, according to claim 2, wherein the alcohol is phenoxyethanol.

4. The optical element for surgical microscopes, according to claim 3, wherein the content of the phenoxyethanol is in a range of 1 wt % to 5 wt %.

5. The optical element for surgical microscopes, according to claim 4, wherein the antireflection coating is formed by layering, from the substrate side, a first high refraction index layer comprising ditantalum pentoxide or hafnium dioxide, a first low refraction index layer comprising silicon oxide, and a second high refraction index layer comprising ditantalum pentoxide or hafnium dioxide.

6. The optical element for surgical microscopes, according to claim 5, wherein the antireflection coating is formed by layering, on the second high refraction index layer, a second low refraction index layer comprising silicon oxide and a third high refraction index layer comprising ditantalum pentoxide or hafnium dioxide.

7. The optical element for surgical microscopes, according to claim 1, wherein the substrate is lanthanum-based glass or synthetic quartz glass.

8. The optical element for surgical microscopes, according to claim 1, wherein coating thickness of the antifouling coating layer is in a range of 1 nm to 10 nm.

9. A method for producing the optical element, for surgical microscopes, according to claim 1, comprising steps of:
forming the antireflection coating on the substrate;
coating solution containing alkoxysilane having acrylic group on the antireflection coating so as to form the antifouling coating layer; and
substituting C—H bonding of carbon of the end of the surface of the antifouling coating layer by C—OSi(OH)₃ by a combustion chemical vapor deposition method using silane based gas.

10. A medical optical device, comprising the optical element for surgical microscopes, according to claim 1.
